(19) United States
(12) Patent Application Publication
(10) Pub. No.: US 2008/0065032 A1
(43) Pub. Date: Mar. 13, 2008

(54) Container for collecting excretions, draining collections, purging ostomies or the like

(76) Inventor: Beniamino Palmieri, Modena (IT)

Correspondence Address: Modiano & Associati, Via Meravigli, 16, Milano, 20123, IT

(21) Appl. No.: 11/629,915

(22) PCT Filed: Jul. 7, 2005
(86) PCT No.: PCT/EP05/53250

§ 371 Date: Dec. 18, 2006

(30) Foreign Application Priority Data
Jul. 8, 2004 (IT) MO2004A000177

(51) Int. Cl. A61F 5/44

(52) U.S. Cl. 604/337; 604/347; 604/352

(57) ABSTRACT

A container for collecting excretions, draining collections and purging ostomies, comprising a bag which is closed along its peripheral edges and on at least one face of which there is a radial arrangement of slits, which form a plurality of independent sectors, each of which is associated with a respective adhesive element intended to adhere to a portion of the skin of a patient which surrounds the exit orifice of the excretion, collection or material to be purged, the hole, which constitutes the center of the radial arrangement, expanding into an opening for the passage of the excretion, collection or material.

### TECHNICAL FIELD

The present invention relates to a container for collecting excretions, draining collections, purging ostomies or the like.

### BACKGROUND ART

In the medical and surgical field, various containers are known which are intended exclusively to collect excretions, urine or feces, or to drain collections or purge ostomies.

For the collection of feces, in addition to disposable diapers or so-called bedpans, containers are known which are substantially constituted by bags which are closed along their perimetric edges and on one face of which there is a circular opening in which the rim is associated with an adhesive element temporarily protected by a removable film and the diameter is such as to surround only the sphincter.

After removing the film, the rim of the opening is made to adhere to the skin that surrounds the sphincter.

In order to apply these containers, instruments are known which are substantially constituted by a bell-shaped retractor, which accommodates internally a disk-like support so that it can slide axially, said support being intended to support the container and being associated with the end of a stem arranged, so that it can slide axially, in a sleeve that lies to the rear of the retractor and ends at the opposite end with a handle.

However, these known containers are not free from drawbacks, including the fact that they do not ensure perfect adhesion of the rim of the opening to the skin that surrounds the sphincter; this rim, as a consequence of a successive contractions and expansions of the sphincter region, in fact tends to come unstuck locally, reducing the seal of the container, causing unwanted external leakage and forming pockets in which the feces collect partly, contaminating the perineal region and thus facilitating the onset of infections, macerations and sores.

Known instruments for applying these containers also have drawbacks, including the fact that they are awkward to use and that they do not adapt to the various anatomical configurations of different patients and therefore do not allow to make the rim of the opening adhere completely to the respective perineal regions.

In order to collect urine, catheters are instead known which are connected to a bag or, for application to male patients, condom-like sheaths are known which lead into a duct which discharges into a bag.

Catheters and sheaths, however, are awkward and unpleasant to use, since in any case they are invasive; moreover, sheaths, by adhering elastically to the penis, cause unpleasant constriction sensations.

Finally, appropriately provided bags are known for purging ostomies, such as colostomies, but they require large flat regions in order to ensure the adhesion of their inlet to the skin and can be applied only to apertures that are not subjected to successive contractions and relaxations, from which they would otherwise detach.

Therefore, the use of these bags is inconveniently limited only to the purging of ostomies.

It is therefore necessary to have different containers depending on the nature of the material to be collected and on the orifice from which it is to be evacuated, and this accordingly entails high costs for the manufacture and management of various store reserves and waste of material.

### DISCLOSURE OF THE INVENTION

The aim of the present invention is to eliminate the drawbacks noted above, by providing a container for collecting excretions, draining collections, purging ostomies or the like which is versatile and flexible in use, hygienic and safe, ensures tightness against leaks and avoids contamination of the skin of patients, preserving them from the risk of infections, macerations and sores.

Further objects of the present invention are to provide a container that is simple, quick and easy to apply, allows to protect medical staff and health workers assigned to its use from the risk of infections or contaminations, and allows them full freedom in performing local procedures on the involved orifice, such as for example the application of medicines, ointments or the like, maneuvers with surgical or diagnostic instruments, washing or others.

Another object of the present invention is to provide an instrument for applying the container that is easy to use and flexible and adaptable to the anatomical configuration of different patients, allowing in any case perfect application of the container.

Within this aim, an object of the present invention is to provide a container that is simple, relatively easy to provide in practice, safe in use, effective in operation, and has a relatively low cost.

This aim and these and other objects that will become better apparent hereinafter are achieved by the present container for collecting excretions, draining collections, purging ostomies or the like, comprising a bag which is closed along its peripheral edges, characterized in that on at feast one face of said bag there is a radial arrangement of slits, which form a plurality of independent sectors, each of which is associated with a respective adhesive element intended to adhere to a portion of the skin of a patient which surrounds the exit orifice of the excretion, collection or material to be purged, the hole, which constitutes the center of said radial arrangement, expanding into an opening for the passage of said excretion, collection or material.

This aim and these objects are also achieved by a device for applying the container to the perineal region, characterized in that it comprises two blades, between which spring means are interposed which are suitable to keep them mutually close and are provided with a handle.

### BRIEF DESCRIPTION OF THE DRAWINGS

Further characteristics and advantages of the present invention will become better apparent from the following detailed description of a preferred but not exclusive embodiment of a container for collecting excretions, draining collections, purging ostomies or the like and of the corresponding applicator, illustrated by way of non-limiting example in the accompanying drawings, wherein:

FIG. 1 is a schematic and partially cutout plan view of a container according to the invention;

FIG. 2 is a schematic plan view of the container according to the invention, with the sectors raised so as to form the passage opening;

FIG. 3 is a schematic perspective view of the tabs for applying the container according to the invention;

FIG. 4 is a schematic plan view of a first alternative embodiment of the container according to the invention;

FIG. 5 is a schematic plan view of a second alternative embodiment of the container according to the invention;

FIG. 6 is a schematic front sectional view of an applicator according to the invention;

FIG. 7 is a schematic side view of the applicator according to the invention;

FIG. 8 is a schematic view of the step for applying the container according to the invention to the perineal region of a patient in order to collect feces;

FIG. 9 is a schematic view of the container according to the invention applied to the perineal region of a patient.

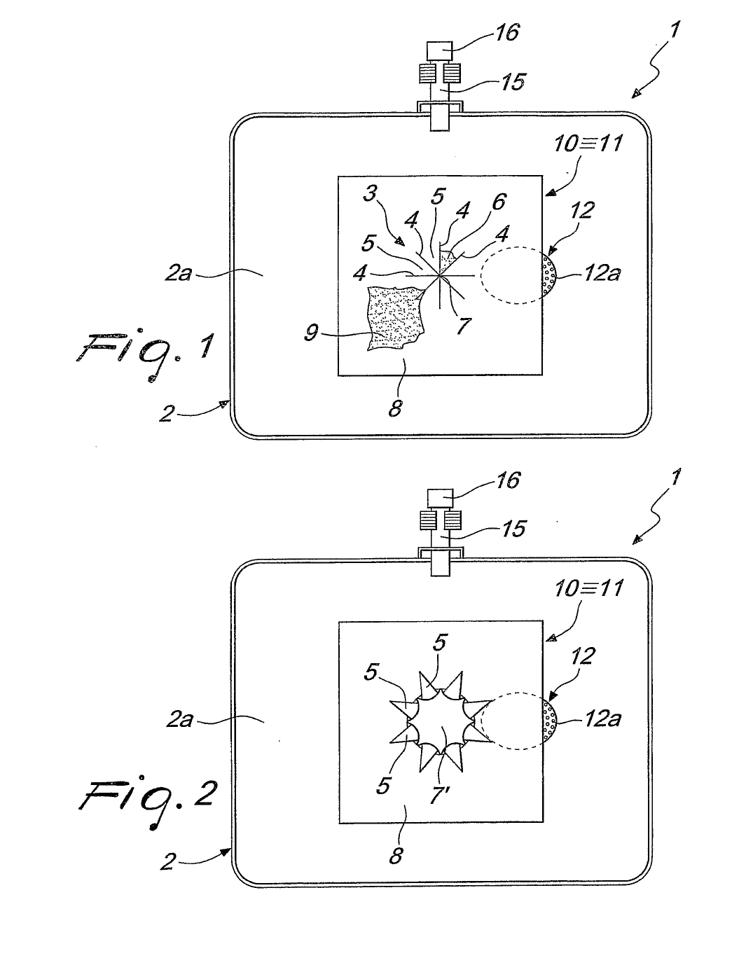

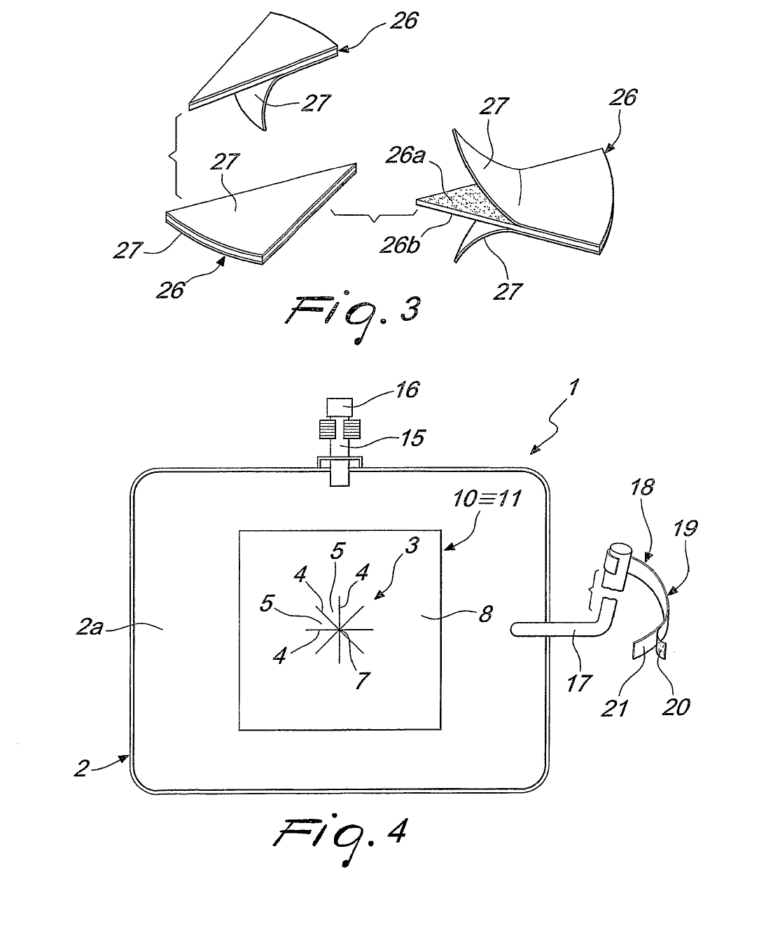

### WAYS OF CARRYING OUT THE INVENTION

With reference to the figures, the reference numeral 1 generally designates a container for collecting excretions, draining collections, purging ostomies or the like.

The container 1 is applied in the medical and surgical field to collect feces and/or urine of immobilized, disabled or incontinent patients, for draining collections from infected cavities, for example in thoracic surgery, in cleaning surgical wounds, such as for example in liver transplants, for hepatic abscesses or abscesses of the abdominal wall that have fistulized externally, for rupturing aneurysms (rexin), in order to recover the blood to be reinfused by autotransfusion, for draining hydrothoraces and chylothoraces, and the like.

Further, the container 1 can be used for wounds complicated by colliquating and suppurating hematomas, for traumatic wounds and lacerated wounds that cannot be sutured, to isolate a surgical incision, whether contaminated or not, and to evacuate necrotic or serohematic or purulent material from cavities, such as for example are from the vaginal cavity, in order to analyze the quality and quantity of the material produced following surgical procedures, infectious processes, hemorrhagic or colliquative processes, without the use of sanitary towels or diapers.

The container 1 comprises a bag 2, which is closed along its peripheral edges and on at least one face 2a of which there is a radial arrangement 3 of slits or cuts 4, which form a plurality of sectors 5, which are mutually independent and each of which is associated, at its surface directed toward the outside of the bag 2, with a respective adhesive element 6, which is intended to adhere to a portion of the skin of a patient that surrounds the exit orifice of the excretion to be collected, the collection to be drained, or the material to be purged.

The radial arrangement 3 forms a hole 7; when the sectors 5 adhere to the skin of the patient, lifting from the plane of arrangement of the face 2a, the hole 7 expands into an opening 7′ for the passage of the excretions, collections or material.

A rim 8 is formed around the radial arrangement 3 and is associated with a respective adhesive element 9, which is suitable to adhere to the peripheral skin of the affected orifice.

The adhesive elements 6 and 9, respectively of the sectors 5 and the rim 8, are covered by a respective protective film 10 and 11, which is detachably associated therewith; advantageously, the two films 10 and 11 are formed in a single sheet.

One face of a flap 12 can be associated on a fraction of the adhesive element 8 and/or 9, and its opposite face 12a, made of non-stick and advantageously porous and breathable material, is designed to cover a portion of the skin of the patient, such as for example the vulva region, protecting it from sticking to the adhesive element 8 and/or 9.

If the container 1 is intended to collect human feces, the hole 7 is arranged proximate to the anus 13 and the sectors 5 are made to adhere to the skin of the glutei 14 that surrounds the anus 13; in this case, the sectors 5 have a height that is substantially equal to 3.5-4 cm, the opening 7′ having an average diameter of approximately 7 cm.

Further, the container 1 comprises a duct 15 for accessing the bag 2, in which one end is associated with the bag 2 and the opposite end is associated with removable closure means, such as for example a screw-on cap 16. Conveniently, it is possible to associate with the duct 15 a one-way valve, not shown as it is of a known type, isolating the operating environment enclosed by the bag 2 and avoiding its contamination from the outside.

It is possible to insert through the duct 15 cannulas for the administration of enemas or washing solutions, syringes or atomizers for applying or spraying disinfectants, medications, ointments or others, surgical instruments such as for example laparoscopes or the like in order to operate for example on polyps, hemorrhoids or condylomas, diagnostic instruments or others.

If the container 1 is intended also to collect urine, it comprises a tubular element 17, in which one end is associated with the bag 2 and the opposite end is suitable to be associated with the urethral orifice and provided with means 18 for adhering to the penis or vulva.

If the container 1 is intended to be applied to a male patient, the adhesion means 18 comprise a tape 19 provided with an adhesive element 20 on one side, said element being covered by a respective protective film 21 of the detachable type and being suitable to be wrapped around the glans and/or penis.

If the container 1 is intended to be applied to a female patient, a collection cup 22 is associated with the free end of the tubular element 17 and is intended to be placed against the vulva, and the adhesion means 18 comprise an adhesive element 23, which is distributed along the upper perimetric rim 24 of the cup 22 and is covered by a respective removable protective film 25.

Conveniently, a spongy element, not shown, suitable to absorb urine and make it percolate into the tubular element 17, can be accommodated inside the cup 22.

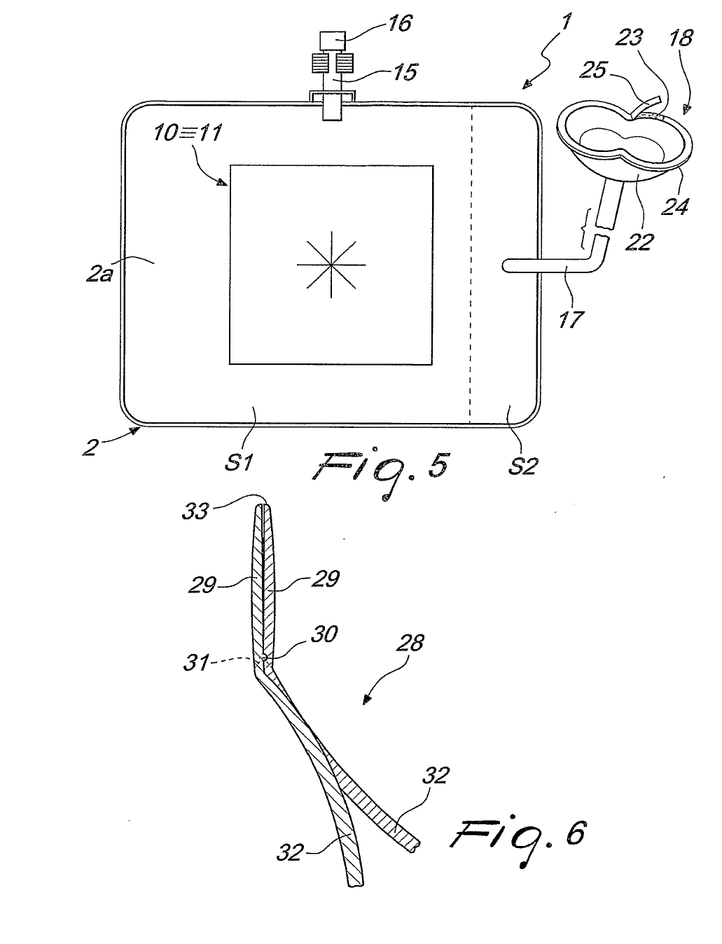

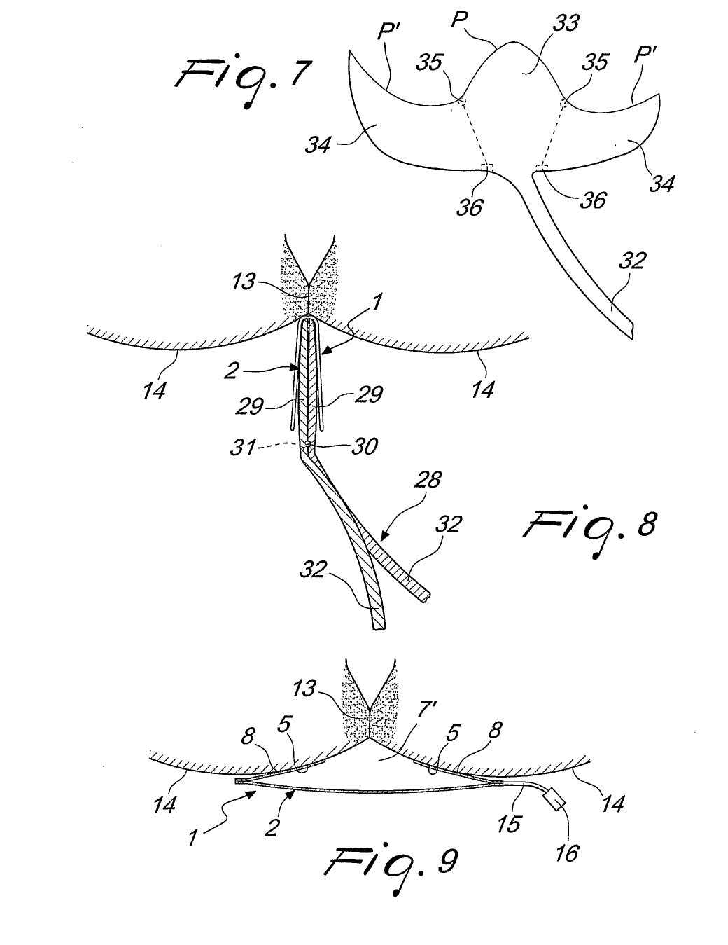

If the container 1 is intended to collect feces and urine, the bag 2 can be divided advantageously into two separate compartments S1 and S2 (FIG. 5), in which one compartment S1 is designed to collect feces and has the radial arrangement 3 of cuts 4 formed thereat, the hole 7 of said radial arrangement being suitable to be arranged at the anus 13, and the other compartment S2 is designed to collect urine and has the tubular element 17 associated thereat.

It is possible to insert in the bag 2 material which adsorbs gases and/or coagulates and/or thickens the collected fluids, or it is possible to aerosolize inside it drugs or other compounds or inject gases.

To facilitate the application of the container 1, the use is provided of a plurality of double-adhesive tabs 26 made of hydrocolloidal resinous material, polyurethane material or the like, having various thicknesses, which are suitable to be interposed between the sectors 5, of which they substantially duplicate the shape and dimensions, and/or the rim 8 and the skin or between the adhesion means 18 and the penis or vulva.

In particular, the tabs 26 are substantially shaped like a triangle, advantageously an isosceles triangle.

A respective removable protective film 27 is applied to each one of the opposite adhesive faces 26a and 26b of each double-adhesive tab 26.

The tabs 26 are applied to the perianal skin or in the vicinity of another orifice before applying the bag 2 and increase the adhesion and seal of the sectors 5 of the bag 2.

A preferred embodiment uses curved or arc-like double-adhesive rods or bars, not shown, in order to increase the adhesion of the sectors 5 to the skin and as a replacement of the tabs 26.

Advantageously, the bars or rods have a length comprised between 0.5 and 1 cm, a width comprised between 0.2 and 4 cm, and a thickness comprised between 0.2 and 0.4 cm.

Such bars or rods are applied to the perianal skin or to the skin that surrounds another orifice and are arranged in one or more concentric rows with their concavity directed toward the anus or other orifice, and the sectors 5 of the bag 2 are then applied onto them. Conveniently, the bars or rods are replaced each time the bag 2 is changed.

In order to apply the container 1 at the perineal region, an applicator 28 is used which comprises two blades 29, which are mutually pivoted about a pivot 30 and between which spring means 31 are interposed which are suitable to keep them mutually close; said blades are provided with a handle 32.

Each blade 29, viewed laterally, comprises a central portion 33, which has a substantially convex profile P and is intended to be arranged at the anus 13; two wings 34 extend in opposite directions from said central portion, have a substantially concave profile P′ and are designed to surround the glutei.

In order to be able to adapt the applicator 28 to the different anatomical configurations of different patients, the wings 34 can be pivoted to the central portion 33 about respective pivots 35, means 36 for fixing their relative position being provided.

Conveniently, the profile P of the central portion 33 protrudes at least partially with respect to the profile P′ of the wings 34 and the handle 32 is inclined with respect to the longitudinal axis of the central portion 33 or with respect to the plane of arrangement of the two blades 29.

The bag 2 is made of impermeable polymeric material, such as polyethylene, polyvinyl chloride, polypropylene, polyvinyl alcohol, polyurethane, or others; the adhesive elements and the double-adhesive tabs 26 are polyurethane-based.

In order to increase the adhesiveness of the adhesive elements 6 and 9 of the bag 2 and of the tabs 26 or of the bars or rods, it is possible to modify their electrostatic charge; in particular, since the skin is characterized by a negative electrostatic charge, it is possible to modify the adhesive of these components with a positive electrostatic charge, for example by dispersing inside it positively charged particles, magnetite or titanium. In this manner, the adhesion to the skin of the adhesive elements 6 and 9 and of the tabs 26 and of the rods or bars is increased by their mutual electrostatic attraction.

Particularly but not exclusively with reference to the application of the container 1 in the perineal region to collect feces, the operation of the invention is as follows.

After removing the films 10 and 11 that cover the adhesive elements 6 and 9 of the sectors 5 and of the rim 8, the bag 2 is rested against the applicator 28 so that the central axis of said bag lies substantially at the end of the blades 29 and half of the bag 2 falls onto the opposite sides of said blades, and the hole 7 is substantially at the apex of the profile P of their central portion 33.

After parting the glutei 14 of the patient, the applicator 28 is inserted, with the bag 2 rested thereon, between said glutei, centering the anus 13 with the central portion 33, and by applying pressure to the handle 32, overcoming the resisting force of the spring means 31, the two blades 29 are moved mutually apart, pressing the sectors 5, and possibly part of the rim 8, against the skin of the patient, to which they adhere.

In case of hypotonus and weakness of the sphincter, it is possible to insert the sectors 5, having an appropriate height, within the anal orifice so as to line its internal walls and skim its ring, facilitating, during expulsion, the entry of the cylinder of feces into the bag 2.

Before applying the container 1, it is possible to arrange around the anus 13 the double-adhesive tabs 26 and/or spray the skin with a fluid adhesive, based for example on gum arabic, so as to improve the adhesion of the sectors 5 and/or of the rim 8 to said skin.

If the tabs 26 are also used, said tabs, having an appropriate height, can be arranged so that their base lies proximate to the rim of the anal orifice and the opposite apex lies so as to adhere to the internal wall of the anal orifice, so as to constitute a guide and a seal for the expulsion of the cylinder of feces into the bag 2, minimizing the possible contamination of the perianal skin.

Once the collection of the material has ended, it is sufficient to detach the bag 2 and fold back the adhesive elements 6 and 9 so as to seal the content of the bag 2 with respect to the external environment without contaminations and without exhalations.

The container according to the invention is also used in surgical procedures and anorectal endoscopic maneuvers, acting both as a device for collecting the material produced in the operated area, to be subjected to possible analysis, without external contaminations and without releasing any exhalations, and as a medication as a replacement of conventional gauzes; the duct for accessing the bag in fact allows to apply locally medications, drugs or others.

The container according to the invention is further used in all cases in which it is necessary to collect in a sterile manner material to be subjected to analysis (bacterial analyses, mycotic analyses, antibiograms, biochemical analyses, chemical-physical analyses, etc), such as for example analysis of occult blood in feces, protecting the collected material against external contaminations and therefore without affecting the results of the analyses performed thereon.

The container according to the invention can be used to collect material from all the cavities connected to movable surfaces or surfaces provided with folds, such as for example the cavities formed in the cervical, mastoid, pelvic, axillary or similar cavities, for collecting for example material from fistulas.

In practice it has been found that the described invention achieves the intended aim and objects.

The invention thus conceived is susceptible of numerous modifications and variations, all of which are within the scope of the appended claims.

All the details may further be replaced with other technically equivalent ones.

In practice, the materials used, as well as the shapes and the dimensions, may be any according to requirements without thereby abandoning the scope of the protection of the appended claims.

The disclosures in Italian Patent Application No. MO2004A000177 from which this application claims priority are incorporated herein by reference.

What is claimed is:

1-33. (canceled)

34. A container for collecting excretions, draining collections and purging ostomies, comprising a bag which is closed along its peripheral edges, wherein on at least one face of said bag there is a radial arrangement of slits, which form a plurality of independent sectors, each of which is associated with a respective adhesive element intended to adhere to a portion of the skin of a patient which surrounds the exit orifice of the excretion, collection or material to be purged, the hole, which constitutes the center of said radial arrangement, expanding into an opening for the passage of said excretion, collection or material.

35. The container of claim 34, further comprising a rim which is formed so as to surround said radial arrangement and is associated with a respective adhesive element and is suitable to adhere to the skin of said patient that is located peripherally with respect to said orifice.

36. The container of claim 35, further comprising at least one protective film, which is associated detachably with the adhesive element of said sectors and/or said rim.

37. The container of claim 35, further comprising a flap provided with a side which is rigidly associated with a fraction of the adhesive element of said sectors and/or of said rim and an opposite side made of non-stick material and intended to preserve a portion of the skin of said patient from adhesive bonding with said adhesive element.

38. The container of claim 34, wherein said sectors have a height that can vary between 0.5 cm and 7 cm, preferably equal to 3.5 cm.

39. The container of claim 34, further comprising at least one duct for access to said bag, in which one end is associated with said bag and an opposite end is associated with removable closure means.

40. The container of claim 39, further comprising a one-way valve associated with said duct.

41. The container of claim 34, further comprising a tubular element in which one end is associated with said bag and an opposite end is suitable to be associated with an urethral orifice and is provided with adhesion means for adhesion to the penis or vulva.

42. The container of claim 41, wherein said adhesion means comprise a tape which is provided, on one side, with an adhesive element and is suitable to be wrapped around the penis.

43. The container of claim 41, wherein said opposite end of the tubular element is associated with a collecting cup, which is intended to be placed against the vulva, said adhesion means comprising an adhesive element, which is distributed along the upper perimetric rim of said cup.

44. The container of claim 43, further comprising a spongy element accommodated within said cup.

45. The container of claim 42, further comprising a protective film which is detachably associated with the adhesive element of said tape or of said rim.

46. The container of claim 41, wherein said bag comprises at least two separate compartments, one of said compartments being intended to collect feces, said radial arrangement of slits being formed thereat, said hole being adapted to be arranged at the anus, said other compartment being intended to collect urine and said tubular element being associated thereat.

47. The container of claim 34, further comprising adsorbent and/or coagulating and/or thickening material accommodated within said bag.

48. The container of claim 41, further comprising at least one double-adhesive tab, which is adapted to be interposed between said sectors and/or said rim and said skin or between said adhesion means and said penis or vulva.

49. The container of claim 48, wherein said tab comprises a protective film, which is detachably associated with each one of its opposite sides.

50. A container for collecting excretions, comprising a bag which is closed along perimetric edges thereof and is associated with an end of a tubular element, an opposite end of which is adapted to be associated with the urethral orifice and is provided with adhesion means for adhesion to the penis or vulva.

51. The container of claim 50, wherein said adhesion means comprise a tape provided on one side of an adhesive element, said tape being designed to be wrapped around the penis.

52. The container of claim 50, wherein said opposite end of the tubular element is associated with a cup which is intended to be placed against the vulva, said adhesion means comprising an adhesive element distributed along the upper perimetric rim of said cup.

53. The container of claim 52, further comprising a spongy element accommodated within said cup.

54. The container of claim 52, comprising a protective film which is detachably associated with the adhesive element of said tape or of said rim.

55. An applicator for applying a container according to claim 34 to a perineal region, comprising two blades, between which spring means are interposed which are adapted to keep said blades mutually close and are provided with a handle.

56. The applicator of claim 55, wherein said blades are mutually pivoted.

57. The applicator of claim 55, wherein each one of said blades comprises a substantially central portion which has a substantially convex profile and from which two wings having a substantially concave profile extend in opposite directions.

58. The applicator of claim 57, wherein said wings are pivoted to said central portion, means for fixing their relative position being provided.

59. The applicator of claim 57, wherein the profile of said central portion protrudes at least partially with respect to a profile of said wings.

60. The applicator of claim 55, wherein said handle is inclined with respect to a longitudinal axis of said central portion.

61. A device for fixing a container according to claim 34, comprising at least one double-adhesive tab, which is adapted to be interposed between said sectors and/or said rim and said skin or between adhesion means and penis or vulva.

62. The device of claim 61, wherein said tab comprises a protective film, which is associated detachably with each one of opposite sides thereof.

63. A method for applying a container according to claim 34, comprising removing said film from said sectors and/or from said rim and/or from a tape and/or from said rim and pressing a respective adhesive element against the skin of the patient that surrounds said orifice.

64. The method of claim 63, comprising removing the protective film from one of the opposite sides of tabs, making the exposed adhesive face adhere to the skin of the patient that surrounds said orifice, and removing a protective film that covers the other side of said tabs before said pressing.

65. The method of claim 63, comprising applying to said skin a fluid adhesive product before said pressing and/or said adhesion.

66. A method for applying a container according to claim 34 in a perineal region, by using an applicator according to claim 55, comprising resting on two mutually close blades said bag so that said side is directed outward and the central axis is substantially located at the end of said blades, the bag falling back on to the sides of said blades, interposing the applicator, with the bag rested thereon, between the glutei of a patient and separating said blades for adhesion of the adhesive element of at least said sectors to the skin that surrounds the anal orifice.
